(19) United States
(12) Patent Application Publication
(10) Pub. No.: US 2022/0411846 A1
(43) Pub. Date: Dec. 29, 2022

(54) METHOD FOR PREDICTING THE VIRULENCE AND PATHOGENICITY OF GRAM-NEGATIVE BACTERIAL STRAINS

(71) Applicants: CENTRE NATIONAL DE LA RECHERCHE SCIENTIFIQUE, Paris (FR); UNIVERSITÉ PARIS-SACLAY, Gif-sur-Yvette (FR)

(72) Inventors: Pierre TISSIERES, Gif-sur-Yvette (FR); Luis AUGUSTO, Orsay (FR); Nadège BOURGEOIS-NICOLAOS, Antony (FR); Richard CHABY, Bagneux (FR); Aude BRETON, Pornic (FR); Florence DOUCET-POPULAIRE, Paris (FR)

(73) Assignees: CENTRE NATIONAL DE LA RECHERCHE SCIENTIFIQUE, Paris (FR); UNIVERSITÉ PARIS-SACLAY, Gif-sur-Yvette (FR)

(21) Appl. No.: 17/778,569

(22) PCT Filed: Nov. 19, 2020
(86) PCT No.: PCT/EP2020/082771

§ 371 Date: May 20, 2022

(30) Foreign Application Priority Data
Nov. 21, 2019 (FR) FR1913060

(51) Int. Cl. C12Q 1/10; C12Q 1/24; G01N 33/92

(52) U.S. Cl. CPC C12Q 1/10; C12Q 1/24; G01N 33/92; G01N 2400/50

(57) ABSTRACT

A method for predicting the pathogenicity and virulence of a strain of Gram-negative bacteria of the Enterobacteriaceae family, wherein the amount of 2-hydroxymyristate and/or 2-hydroxymyristic acid present in the lipopolysaccharides of the bacteria is identified and measured, the amount of 2-hydroxymyristate and/or 2-hydroxymyristic acid is compared with a reference value, and wherein it is concluded that the strain is virulent if the amount of 2-hydroxymyristate is greater than the reference value. Also, the use of the 2-hydroxymyristic acid ester as a marker of pathogenicity and virulence of a Gram-negative bacterial strain and an in vitro diagnosis kit implementing this marker.

The invention relates to the field of infections by Gram-negative bacteria of the enterobacteriaceae family, in particular by Enterobacter cloacae, and the means for predicting their virulence and/or their pathogenic character. The invention particularly relates to new biomarkers and to their use, as well as to the devices allowing their use in prediction methods, in particular clinical prediction.

Infections with the Enterobacter cloacae bacterium are responsible for severe infection in humans that can progress to septic shock and to the death of the patient. Some population groups, such as elderly patients, immunocompromised patients and prematurely born infants are at high risk of contracting this infection. In premature infants, infection with Enterobacter cloacae is fatal in 80% of cases and occurs epidemically.

Species of the Enterobacter cloacae complex, Gram-negative bacteria of the Enterobacteriaceae family, are generally isolated from soil, plants, and the intestines of mammals and insects. As a facultative anaerobe, they have the ability to survive in a variety of environments, in particular in dry soil, water pipes, and metal or plastic medical equipment. E. cloacae complex is a common contaminant of central intravascular catheters, of biomedical equipment and can be an opportunistic pathogen in immunocompromised humans and newborns responsible for nosocomial infections. The E. cloacae complex includes six different species: Enterobacter asburiae, Enterobacter cloacae, Enterobacter hormaechei, Enterobacter kobei, Enterobacter ludwigii and Enterobacter nimipressuralis.

In humans, the E. cloacae complex (E. cloacae) forms part of the normal intestinal microbiota and generally is not a primary pathogen. However, some strains become virulent, in particular in hospital environments: the E. cloacae complex has become one of the most commonly encountered nosocomial pathogens in neonatal, pediatric and adult intensive care units. The factors associated with the virulence of Enterobacter cloacae are currently poorly understood.

Wild strains of Enterobacter cloacae are susceptible to third generation cephalosporins, fourth generation cephalosporins, aminoglycosides, quinolones, sulfonamides, tetracyclines and chloramphenicol. However, the emergence and dissemination of strains that are multi-resistant to antibiotics in hospital environments requires the use of last-resort antibiotics. In the specific case of neonatal infections, a combination of a penicillin or a 3^{rd} generation cephalosporin with another class of antibiotic, in particular aminoglycosides (gentamycin), is conventionally used as a first-line of treatment. If the treatment is ineffective, second-line antibiotic therapy is implemented with other combinations based on penicillins and beta-lactamase inhibitors, carbapenems, polymixins and/or tigecycline. In the case of resistant strains, third- or fourth-line antibiotic treatments will be necessary in order to eradicate the infection.

The therapeutic pressure caused by the treatment of Enterobacter cloacae contributes to the emergence of multi-resistant strains. In the case of virulent strains, the infections are likely to lead to the death of the patient within a very short time, sometimes around 48 hours. Therefore, it is not possible, in these cases, to wait for the patient's reaction to a first-line treatment in order to adapt it in the event of a lack of efficacy. The extreme severity of these infections in vulnerable population groups leads to a therapeutic scheme with immediate recourse to treatments with third- or fourth-line antibiotics, as soon as the presence of Enterobacter strains is detected. It is only a posteriori that the susceptibility to different antibiotics of the strains identified during sampling may or may not be confirmed using conventional techniques. However, a significant number of patients can be asymptomatic carriers, which does not require treatment.

Furthermore, although not specific to E. cloacae, the extensive use of broad-spectrum antibiotics is responsible for the development of resistance and the selection of pathogenic strains. It results in a change in the epidemiology of neonatal infections with the emergence of serious enterobacteria infections, including Escherichia coli, Klebsiella pneumoniae and Enterobacter cloacae. It is therefore desirable to reserve the use of second-line antibiotic therapies for strains that are virulent and/or resistant to first-line antibiotics responsible for invasive infections.

In addition to the patient's own carriage of Enterobacter cloacae strains, contamination of equipment (for example, incubators for prematurely born infants) is a potential source of patient infection. Therefore, it is also important to determine whether this can be treated using common methods or whether the destruction and the replacement of the equipment is necessary in view of the pathogenicity of the strains.

It is essential to be able to differentiate strains with a high risk of human pathogenicity from those responsible for asymptomatic carriage in order: 1) to initiate appropriate treatments as quickly as possible for pathogenic strains before the invasive infection, septic shock and death of the patient; and 2) not to unnecessarily treat non-pathogenic strains and therefore generate unnecessary antibiotic selection pressure.

Therefore, a need exists to rapidly detect and identify the pathogenic strains requiring an appropriate treatment to be established before the patient dies.

A need also exists to identify non-pathogenic strains and to avoid unnecessary treatment.

Within the scope of the present invention, a correlation between a specific structure of the lipopolysaccharide (LPS) and the pathogenicity of nosocomial strains of enterobacteriaceae, in particular of antibiotic resistant E. cloacae, has been demonstrated.

The invention therefore allows a rapid and reliable technique to be established for detecting the pathogenicity and/or the virulent nature of clinical strains of enterobacteriaceae such as Enterobacter cloacae. This is achieved by the characterization of a specific marker, present in the LPS, and more particularly in the lipid A of the bacteria.

Lipopolysaccharide (LPS) is a major component of the outer surface of Gram-negative bacteria. LPS is made up of three entities: (i) lipid A, has a hydrophobic character; (ii) O antigen (for “Ohne Kapsel”), located on the distal part of the LPS, with a polysaccharide nature, has a hydrophilic character; and (iii) the nucleus (or “core”) with a polysaccharide nature represents the bridge between the other two parts. Lipid A, embedded in the outer membrane, represents the proximal part of the LPS, the nucleus represents its middle part, and the O antigen represents its “free” distal part in the external environment. In enterobacteriaceae, lipid A is highly preserved and the nucleus varies very little, while the O antigen is the hypervariable region. Several biological activities and roles have been associated with LPS, including the endotoxin activity carried by lipid A and the antigenic specificity of the bacterial strain carried by O antigen.

The structure of lipid A is sufficiently preserved among all Gram-negative bacteria. Among bacteria of the same family, such as the Enterobacteriaceae family, the structure of lipid A is even almost identical. In E. coli, lipid A is made up of a D-glucosamine disaccharide linked by the β-1.6 bond and phosphorylated at position 1 and 4′. This disaccharide is acylated at position 2, 3, 2′ and 3′ with four b-hydroxymyristoyl groups. Two other fatty acid chains: a laurate residue (12c) and a myristate residue (14) esterified at their non-reducing end can bind on the first two hydroxymyristoyl residues (Raetz, 1990).

The role of LPS in the resistance of Gram-negative bacteria to antibiotics and antimicrobial agents is directly related to the barrier that the LPS forms on the outer surface of the bacterial membrane. Hydrophilic antibiotics, such as β-lactams, use porins to penetrate inside the bacterial cell, while other drugs must destabilize this barrier, which is made rigid due to strong interactions between the LPS molecules. Consequently, changes in the lipid A region of the LPSs can alter the penetration ability of some antimicrobial agents. For example, adding 4-aminoarabinose or phosphoethanolamine residues on the phosphate groups of lipid A reduces the negative charge of the LPSs, thus decreasing the repulsion between adjacent LPS molecules. Furthermore, adding a palmitate to lipid A increases the hydrophobic interactions between neighboring LPS molecules.

In Gram-negative bacteria, increased virulence can be due to increased resistance to different cationic antimicrobial peptides (CAMPs) produced by the host, such as defensins, human neutrophil peptide (HNP-1), human cationic protein 18 (hCAP18 or LL-37), and kinocidin14. The main mechanisms of resistance to CAMPs involve modifications of the LPSs by adding 4-amino-4-deoxy-L-arabinose or phosphoethanolamine, which decreases the negative charge of lipid A. The operons encoding the enzymes involved in these modifications are arnBCADTEF and pmrCAB. Activation of the LPS-modifying genes is often mediated by PmrA/PmrB and PhoP/PhoQ, species-interconnected two-component regulatory systems (TCS). For example, in Escherichia coli, Salmonella enterica or Klebsiella pneumoniae, the phosphorylated form of PhoP can stimulate the expression of PmrD, which in turn activates PmrA by promoting the transcription of the arnBCADTEF and pmrCAB operons. With respect to E. cloacae and its resistance to CAMPs, it has recently been demonstrated that an artificial operon is involved, but, unlike E. coli, Salmonella or Klebsiella, only PhoP/PhoQ (and not PmrA/PmrB) seems to play a role.

The increase in the number of infections contracted in a hospital environment in intensive care units due to E. cloacae and their significant mortality shows that there is an urgent need to identify markers of pathogenicity.

Due to the fulminant evolution of Enterobacter cloacae infections and the septic shocks that they cause, the work carried out within the scope of the invention aimed to study the structures of the lipopolysaccharides of clinical strains responsible for death or asymptomatic carriage; they were able to establish a correlation between some of these structures and the clinical picture, in particular with the survival of infected patients.

The particular aim of the invention is to propose an in vitro detection test for identifying the pathogenic strains of enterobacteriaceae, such as those of Enterobacter cloacae responsible for invasive infection and septic shock and associated excess mortality.

The aim of the present invention is a method for predicting the pathogenicity of a strain of Gram-negative bacteria of the Enterobacteriaceae family, wherein the amount of 2-hydroxymyristic acid or of one of its esters present in the lipopolysaccharide of bacteria is measured, said amount of 2-hydroxymyristic acid or 2-hydroxymyristic acid esters is compared with a reference value, and wherein it is concluded that the strain is pathogenic if the amount of 2-hydroxymyristic acid or 2-hydroxymyristic acid esters is greater than the reference value.

The particular aim of the present invention is a method for predicting the pathogenicity of a strain of Gram-negative bacteria belonging to the genus Enterobacter, Escherichia, Klebsiella, Pseudomonas, Salmonella, Serratia or Yersinia, and in particular to the genus Enterobacter, Escherichia, Pseudomonas, Serratia or Yersinia, wherein the amount of 2-hydroxymyristic acid or of one of its esters present in the lipopolysaccharide of the bacteria is measured, said amount of 2-hydroxymyristic acid or of 2-hydroxymyristic acid esters is compared with a reference value, and wherein it is concluded that the strain is pathogenic if the amount of 2-hydroxymyristic acid or 2-hydroxymyristic acid esters is greater than the reference value.

According to another aspect, the aim of the invention is a method for predicting the virulence of a strain of Gram-negative bacteria of the Enterobacteriaceae family, and in particular of the genus Enterobacter, Escherichia, Klebsiella, Pseudomonas, Salmonella, Serratia or Yersinia, in particular to the genus Enterobacter, Escherichia, Pseudomonas, Serratia or Yersinia, wherein the amount of 2-hydroxymyristic acid or of one of its esters present in the lipopolysaccharide of the bacteria is measured, said amount 2-hydroxymyristic acid or 2-hydroxy-myristic acid esters is compared with a reference value, and wherein it is concluded that the strain is pathogenic if the amount of 2-hydroxymyristic acid or 2-hydroxymyristic acid esters is greater than the reference value.

The method for predicting the pathogenicity and/or the virulence of a strain of Gram-negative bacteria of the Enterobacteriaceae family, and in particular of bacteria of the genus Enterobacter, Escherichia, Klebsiella, Pseudomonas, Salmonella, Serratia or Yersinia, and in particular of the genus Enterobacter, Escherichia, Pseudomonas, Serratia or Yersinia, is based on the qualitative and quantitative measurement of 2-hydroxymyristic acid or one of its esters such as 2-hydroxymyristate present in bacterial lipopolysaccharides. Comparing the amount of 2-hydroxymyristate and/or of 2-hydroxymyristic acid with a reference value allows the virulent and/or pathogenic nature of the studied strain to be determined.

Thus, the prediction method according to the invention can comprise identifying 2-hydroxymyristic acid or one of its esters present in the lipopolysaccharides of the bacteria.

The reference value can be equal to 0, or can be that of a negative control subjected to the same determination. It will thus be adapted by a person skilled in the art according to the susceptibility of the method and the detection threshold of the measured compounds of interest.

The reference value will thus, in some embodiments of the invention, be set to 0.001, or to 0.01.

Advantageously, the bacterium is a strain of the genus Enterobacter, in particular a bacterium belonging to Enterobacter cloacae complex, and selected from Enterobacter asburiae, Enterobacter cloacae, Enterobacter hormaechei, Enterobacter kobei, Enterobacter bugandensis, Enterobacter roggenkampii, Enterobacter ludwigii and Enterobacter nimipressuralis.

In the description, unless otherwise specified, the terms Enterobacter cloacae (or E. cloacae) by default denote a strain belonging to Enterobacter cloacae complex and selected from one of these sub-species: Enterobacter asburiae, Enterobacter cloacae, Enterobacter hormaechei, Enterobacter kobei, Enterobacter bugandensis, Enterobacter roggenkampii, Enterobacter ludwigii and Enterobacter nimipressuralis.

According to another aspect of the invention, the bacterium is a strain of enterobacteria pathogenic for humans, belonging to one of the following species: Escherichia coli, Klebsiella pneumoniae, Pseudomonas aeruginosa and Yersinia pestis.

A pathogenic strain is understood to mean a strain capable of causing an invasive infection, a septic shock or even the death of the patient in connection with this infection, within a short period of time, from a few hours to a few days, in particular within a period of less than 5 days, in particular, which can lead to septic shock and/or the death of the patient within a period that is less than or equal to 48 hours.

To the knowledge of the inventors, it has never been shown that the presence of 2-OH myristate is a prediction marker for the ability of a strain of Gram-negative bacteria, in particular Enterobacter cloacae complex, to cause the death of the patient carrying said strain.

A virulent strain is understood to mean a strain exhibiting characteristics enabling it to escape the host's defense mechanisms, in particular cationic antimicrobial peptides, phagocytosis and the cytokine response.

In particular, a bacterial strain is understood that exhibits resistance to one or more antimicrobial agents such as antibiotics selected from third-generation cephalosporins, penicillins, carbapenems, aminoglycosides, quinolones, sulphonamides, tetracyclines and chloramphenicol. Such strains can particularly carry extended-spectrum beta-lactamases (ESBL). The virulent strain also can be a strain resistant to antimicrobial agents that are the cationic antimicrobial peptides produced by the organism to defend against infections, such as defensins, human neutrophil peptide (HNP-1), human cationic protein 18 (hCAP18 or LL-37), and kinocidin14 derived from human platelets.

According to one of its aspects, the prediction method is a method for the in vitro diagnosis of the pathogenicity of clinical strains of the Enterobacteriaceae family, in particular of strains of bacteria of the genus Enterobacter, Escherichia, Klebsiella, Pseudomonas, Salmonella, Serratia or Yersinia, and in particular to the genus Enterobacter, Escherichia, Pseudomonas, Serratia or Yersinia, in particular Enterobacter cloacae as defined in the present description.

According to another one of its aspects, the prediction method of the invention is a method for predicting the resistance of strains of Enterobacter cloacae to antibiotics and/or to antimicrobial peptides.

According to one of the embodiments of the prediction method according to the invention, the amount of 2-hydroxymyristate is compared with the amount of 3-hydroxymyristate present in the lipopolysaccharide of the bacteria, and if the ratio of 2-hydroxymyristate to 3-hydroxymyristate is greater than or equal to 0.01, it is concluded that the strain is pathogenic.

The invention will be particularly implemented in order to characterize bacteria present in a sample, in particular a biological sample, skin and/or hygiene samples, i.e., any microbiological sample of medical equipment and originating from the environment.

The biological sample can be obtained from a mammal, preferably from a human being. A biological sample can be taken from any sample taken from a subject suspected of carrying strains of Enterobacteriaceae, in particular Enterobacter cloacae. Biological samples can be cited, in a non-limiting manner, that are selected from blood, serum, plasma, cerebrospinal fluid (CSF), ascites and pleural fluid, mucus, stools and urine or any other secretion or excretion. The sample particularly can be obtained from a sample from the respiratory tract and/or from at least part of the pharynx such as the nasopharynx (or cavum), from a sample from the skin obtained by a skin smear or a vaginal sample, for example.

The prediction method according to the invention is particularly suitable for detecting and characterizing strains of Enterobacter cloacae present in vulnerable patients such as newborns, in particular in prematurely born infants, immunosuppressed or immunocompromised patients, polymorbid patients. In all these patients, it is particularly useful to know the nature of the pathogenicity and/or the virulence of the strain at an early stage. This enables a quicker decision to be made with respect to the type of antibiotics to be administered, without waiting for the first symptoms of infection. The fulminant and devastating nature of this type of infection requires anticipation of the treatments to be applied.

Within the context of neonatal infections, the method also can be implemented on maternal biological samples, in particular microbiological screening samples, particularly originating from the vaginal canal, for example, a vaginal smear, from the placenta, or from amniotic fluid. Indeed, the contamination of the newborn can be of maternal origin (per partum, feto-maternal infection).

If a pathogenic strain of Enterobacter cloacae is identified in the mother at the end of gestation or at the time of delivery, particularly in the vaginal canal, on placenta smears or on a culture of amniotic fluid, preemptive initiation of appropriate antibiotic treatment can be contemplated and can justify increased monitoring of the newborn.

The prediction method can be applied to bacteria of the genus Enterobacter cloacae, and in particular Enterobacter cloacae present in subjects exhibiting various types of infections, in particular in elderly and/or immunocompromised patients, and intensive care patients. It is known that Enterobacter cloacae can be involved in infections of the blood, the respiratory tract, osteo-articular pathologies, urinary infections or skin infections, for example: the prediction method can be implemented for strains originating from a subject exhibiting one of these pathologies.

According to another aspect of the invention, the prediction method can be implemented for bacteria originating from a hygiene or environmental sample, in particular a sample taken from equipment or apparatus, such as hospital and/or biomedical equipment. Enterobacter cloacae can indeed contaminate the equipment that is used, which then becomes a source of contamination for the patients who will be in contact therewith. This is the case, for example, for catheters, probes, or incubators in which newborns are placed.

In the case of the detection of a strain of Enterobacter cloacae on equipment, it is important to determine whether it is a pathogenic strain requiring special treatment (or even destruction of the contaminated equipment).

According to one of its embodiments, the prediction method comprises a prior step of isolating bacteria from a sample, in particular from a biological sample, or from a hygiene or environmental sample.

For example, a sample likely to contain Enterobacter cloacae is collected, and a first step of identifying the species is carried out according to techniques that are known to a person skilled in the art.

The strain is then isolated and cultured on a suitable medium.

Advantageously, the method then comprises a washing step in order to separate the bacteria from the culture medium and any contaminants. The method then comprises the following steps:

a) isolating Gram-negative bacteria of the genus Enterobacter from a sample;

b) cultivating the bacteria isolated in step a) in vitro on a suitable medium;

c) recovering the bacteria from the culture medium;

d) washing the bacterial cells and separating the solid part containing the bacterial cells from the liquid part;

e) measuring the amount of 2-hydroxymyristic acid present in the lipopolysaccharide of the bacteria obtained in step d;

f) comparing said amount of 2-hydroxymyristic acid with a reference value;

g) predicting the pathogenicity of the bacteria present in the sample if the amount of 2-hydroxymyristic acid determined in step e) is greater than the reference value.

According to an alternative embodiment of the invention, hydrolysis of the bacterial cells is carried out before step e).

According to another alternative embodiment of the invention, a step of freeze-drying the bacteria is carried out before step d).

According to another embodiment of the invention, the method comprises the following steps:

a) isolating Gram-negative bacteria of the enterobacteriaceae family, in particular of the genus Enterobacter, from a sample;

b) cultivating the bacteria isolated in step a) in vitro on a suitable medium;

c) recovering the bacteria from the culture medium;

d) extracting the lipopolysaccharides present in the bacteria recovered in step c);

e) measuring the amount of 2-hydroxymyristic acid present in the lipopolysaccharide obtained in step d;

f) comparing said amount of 2-hydroxymyristic acid with a reference value;

g) predicting the pathogenicity of the bacteria present in the sample if the amount of 2-hydroxymyristic acid determined in step e) is greater than the reference value.

Advantageously, the Gram-negative bacteria are bacteria of the species Enterobacter cloacae.

The presence and the amount of hydroxymyristic acid, in particular of 2-hydroxymyristic acid, in the bacteria can be measured using methods that are known to a person skilled in the art, such as gas chromatography, gas liquid chromatography or HPLC. According to some embodiments, the bacterial sample will be treated before its chromatography analysis, for example, by solid phase extraction.

Determining the presence of 2-hydroxymyristic acid (or its esters), and measuring the amount that is present can be carried out directly on the cultured strains, after separation from the culture medium. This determination can, according to an alternative embodiment of the invention, be carried out on the lipopolysaccharides, which have been previously extracted from the bacterial culture.

According to an advantageous embodiment, the amount of 2-hydroxymyristic acid and/or its esters such as 2-hydroxymyristate is determined using mass spectrometry.

A mass spectrometer has 3 parts: (i) an ion source, where the ions are produced in the gas phase from solid, liquid or gaseous states; (ii) one or more analyzers, in which the ions are manipulated (transported, rotated, sorted, selected, fragmented, etc.); and (iii) a detector, which counts the ions and amplifies their signals or records the image of a current induced by the movement of the ions.

Finally, a computer system that gathers all the data from these three elements in order to generate a mass spectrum. The mass spectrometer can include other elements, including separation systems such as interfaces with chromatography systems.

Depending on the nature of the ion source and the analyzer, various types of spectrometry are defined. Analyzers of the FT-ICR (Fourier Transform Ion Cyclotron), Orbitrap, ion trap, triple quadrupole and TOF (Time Of Flight) type can be cited in a non-limiting manner. Similarly, ion sources such as Electrospray, MALDI (Matrix Assisted Desorption Ionization), electron impact, APPI, FAB-MS, chemical ionization and magnetic sector can be cited.

The resolution, i.e., the ability to separate two peaks, will vary depending on these sources and analyzers and will be adapted by a person skilled in the art.

The interfaced chromatography systems can comprise any chromatography system, such as gas chromatography (GC), liquid chromatography (LC), or ion mobility (optionally combined with the GC and/or LC methods). According to some embodiments of the invention, the mass spectrometry system is a GC-MS or LC-MS system.

The αhydroxymyristic acid can particularly be identified in two different ways:

According to one embodiment, the lipopolysaccharides (LPS) are extracted from the cultivated strains. The structures of lipid A are analyzed, for example, using the MALDI-TOF technique or any other technique known to a person skilled in the art, in order to determine the presence of 2-hydroxymyristic acid or of 2-hydroxymyristate. As a technique that can be used, TLC (“Thin Layer Chromatography”), Maldi TOF (MS), Maldi TOF/TOF (MS/MS), ESI (Electrospray), GC (Gas Chromatography), GC/MS (Gas Chromatography/Mass Spectrometry), LC/MS (Liquid Chromatography/Mass Spectrometry) and NMR (Nuclear Magnetic Resonance) can be cited in a non-limiting manner.

According to another embodiment, the whole bacteria are treated and then analyzed directly without a GC-MS chromatography type extraction step or any other technique known to a person skilled in the art, in order to determine the presence of 2-hydroxymyristic acid or 2-hydroxymyristate. The treatment of the bacteria can particularly comprise a step of eliminating phospholipids, for example, during a step of washing with one or more solvents and a step of hydrolysis of the bacterial cell. Optionally, the bacteria can be freeze-dried after washing.

Mass spectrometry is an analytical technique used to identify various types of ionized molecules by measuring their mass/charge ratio (m/z). Technology of the MALDI-TOF (or Matrix-Assisted Laser Desorption Ionization-Time Of Flight) type requires a prior step of crystallization, on an inert support, of the sample in a matrix. The complex thus formed is bombarded by a laser beam emitting in the absorption zone of the matrix. The irradiation of the crystalline mixture leads to the desorption of characteristic ions of the sample (MALDI). The ionized molecules are then accelerated in an electric field and directed toward an analyzer separating the ions according to their time of flight (TOF), which is proportional to the mass and the charge. Thus, depending on their m/z ratio, the ions reach the detector fairly quickly, where they are then converted into an electrical signal, which will be amplified and then analyzed. The MALDI-TOF MS type technique is conventionally used to quickly identify microorganisms in the species by analyzing their total proteins, by comparing the obtained spectra with reference spectra. The identification can be carried out from whole microorganisms.

In lipid A of E. cloacae strains or pathogens, small amounts of α-hydroxymyristic acid (2OH—C14) replace the C14:0 secondary chain at position 2 or at position 3′.

The formation of 2-hydroxymyristate requires the action of enzymes encoded, among others, by the IpxO gene. However, it should be noted that 2OH—C14 can be absent, even in the presence of LpxO, if a counterpart enzyme to LpxL2 is absent. Indeed, LpxO only modifies a secondary myristate group previously transferred by LpxL2. Consequently, the IpxO gene alone cannot entirely explain the presence or absence of the α-hydroxymyristate in the LPS. Variants of IpxO encoding different enzymes in terms of specificity possibly can be involved. The results show that 2OH—C14 is a secondary substituent at position 2 in some molecular species (FIG. 3A) and 3′ in others (FIG. 3B).

The inventors have shown that in the strains of the Enterobacter cloacae complex, the DNA encoding LpxO is present, either in the genome of the bacterium or carried by a plasmid.

Analyzes of the lipid A structures using MALDI-TOF, followed by analyzes of their fatty acid content using GC-MS, have shown an indisputable statistical correlation between the presence of 2-hydroxymyristic acid in the LPS of the strains of Enterobacter cloacae and the pathogenicity of the strains, resulting in patient mortality.

By contrast, patient mortality correlates neither with other structural features of endotoxins nor with phenotypic markers such as ERIC-PCR profiles or the AmpC expression.

Thus, in a study carried out on a panel of 36 infants within the scope of the invention, it should be noted that the six babies who died in the absence of 2-hydroxymyristic acid (or 2OH—C14) in their isolates died from pathologies unrelated to E. cloacae infection: pulmonary hemorrhage, meningitis caused by another germ, digestive perforation and enterocolitis, intracerebral hemorrhage.

The invention therefore provides a biomarker of the pathogenicity and/or of the virulence of strains of Enterobacter.

A further aim of the invention is the use of 2-hydroxymyristic acid or its esters as a virulence and/or pathogenicity marker of a Gram-negative bacterial strain of the Enterobacteriaceae family, in particular of the genus Enterobacter, Escherichia, Klebsiella, Pseudomonas, Salmonella, Serratia or Yersinia and in particular of the genus Enterobacter, Escherichia, Pseudomonas, Serratia or Yersinia and in particular of Enterobacter cloacae complex.

This marker can be used in in vitro diagnosis methods.

For this reason, a further aim of the invention is a kit for diagnosing the pathogenicity and/or virulence of strains of enterobacteriaceae (in particular of Enterobacter cloacae complex), with the kit comprising means for measuring the concentration of 2-hydroxymyristate (or 2-hydroxymyristic acid) in the LPS of a bacterial isolate.

The kit can contain, for example, organic solvents useful for the preparation of samples before analyzing their fatty acid content. These solvents in particular can be selected from hexane, chloroform, methanol and their mixtures together and/or with water in all proportions.

By way of an example, a protocol for the GC/MS Diagnosis is described hereafter:

1) Directly from Bacteria:

For the treatment and prior preparation of samples before branching. Washing can be carried out using hexane or a mixture of chloroform/methanol/water, etc.

1 A) Proposed Bacteria Washing Kit:

1 glass tube containing 2 ml of hexane;

1 empty glass tube with plug;

1 Pyrex glass screw tube.

Proposed Protocol for Washing Bacteria:

Add the bacteria (lyophilized or not lyophilized) to the Pyrex glass tube and 500 μl of hexane;

Shake for 2 min;

Centrifuge for 10 min at 2000 g;

Eliminate the organic phase in the empty glass tube acting as a bin;

Re-extract the pellet in the same way 2 more times;

Dry the bacterial pellet.

1 b) Hydrolysis and Branching Kit:

1 tube containing 700 μl of anhydrous methanol;

1 tube containing 100 μl of acetyl chloride;

1 vial containing 10 μg of standard methylated AG C20 (eicosanoic acid);

1 tube containing 1.5 ml of ethyl acetate;

1 tube containing 200 μl of sterile and endotoxin free H2O;

Empty vial with insert and septum for GC/MS (adapted to the type of device).

Proposed Protocol for Hydrolysis and Branching (Methylation):

To the dry Pyrex tube containing the pellet after washing:

Add 520 μl of anhydrous methanol;

Add 5 μl of standard C20 at 1 μg/μl after having reconstituted the standard vial with 10 μl of ethyl acetate;

Very slowly add 70 μl of acetyl chloride, a small amount at a time while shaking the Pyrex tube;

Heat the tube to 85° C. (time to be defined for the kit);

Cool the tube following the treatment time;

Add 500 μl of ethyl acetate;

Evaporate the whole;

Add 100 μl of H₂O and 600 μl of ethyl acetate;

Centrifuge for 10 min at 2000 g;

Recover the upper organic phase and dry it;

Take up the material with 50 μl of ethyl acetate and transfer it to the vial with the insert in order to inject 5 μl by GC/MS.

2) From any Previously Extracted LPSs:

The same protocol and previous kits are adapted without the washing step.

For branching, a mixture of anhydrous methanol and acetyl chloride (10/1.5) can be used. However, the ratio can be adjusted; it allows both the hydrolysis of fatty acids from the LPS and the branching thereof in one step. It also can be branched using other methods.

For GC/MS injection: The solvent used must allow take up of the sample and must be highly volatile. Ethyl acetate, chloroform, hexane or other compounds can be used.

As mentioned above, the LpxO enzyme or the DNA encoding LpxO can also form a marker of the pathogenicity of a strain of E. cloacae.

According to another aspect, the invention relates to a method for in vitro diagnosis of the pathogenicity of a strain of bacteria of the genus Enterobacter, in particular of strains of E. cloacae complex, wherein the presence of nucleic acid encoding the LpxO enzyme in the genetic material of the bacteria is determined.

Said nucleic acid can be present in the genome of the bacterium (genomic DNA or RNA), or in a plasmid.

The invention thus relates to a method for predicting the pathogenicity and/or the virulence of a strain of bacteria of the genus Enterobacter, in particular of strains of E. cloacae complex, wherein the presence of DNA encoding the LpxO enzyme is detected in the genomic or plasmid DNA of the bacteria.

When the nucleic acid encoding LpxO is present in the genetic material, in particular in the genomic or plasmid DNA of the bacterium, it is concluded that the strain is pathogenic.

The methods and procedures will be implemented using techniques that are known to a person skilled in the art, after extraction of the genomic and plasmid nucleic acid, PCR amplification of the sequences defined by specific primers of the gene encoding LpxO. This molecular diagnosis technique can be adapted to various systems, for example, chip, cartridge/pouch or PCR/Q-PCR.

The invention also relates to diagnosis kits for detecting and identifying DNA encoding LpxO.

### BRIEF DESCRIPTION OF THE FIGURES

The invention will be better understood from reading the following examples. In these examples, reference will be made to the following figures:

FIG. 1 Negative-ion MALDI/TOF mass spectrometry of lipid A of the E. cloacae type E strain with cephalosporinase overproduction. P, L-Ara4N, and C16 represent m/z movements corresponding to the phosphate, 4-amino-4-deoxy-L-arabinose, and palmitate substituents, respectively. The marked peaks represent the species where C14:0 is replaced by C12:0 (♦) or 2-hydroxymyristate (•);

FIG. 2 Negative-ion MALDI/TOF mass spectrometry of the E. cloacae strain lipid A, untreated or hydrolyzed with HCl;

FIG. 3 Structure of the main molecular species of the E-type lipid A of E. cloacae. Structures A and B are present in almost equal amounts. [x] minor substituents: L-Ara4N/PO3H2; [y] minor variants: C12:0/C13:0/2OH—C14; [z] minor substituent: C16:0;

FIG. 4 Gas chromatogram of the LPS fatty acid methyl esters of the strain of E. cloacae H7i;

FIG. 5 Survival analysis according to Kaplan-Meyer as a function of the presence of LPS carrying 2OH—C14. The solid line corresponds to the strains for which 2-hydroxymyristic acid is present in the LPS. The dashed line corresponds to strains for which 2-hydroxymyristic acid is absent in the LPS;

FIG. 6 Survival test of strains of Enterobacter cloacae complex expressing 2-hydroxymyristate and not expressing 2-hydroxymyristate, after 4 hours of exposure to different concentrations of polymyxin B.

### EXAMPLE 1

18 patients from the neonatal intensive care unit of the Antoine Béclère hospital, Assistance-Publique Hôpitaux de Paris (Paris Public Health Care Hospitals) (Clamart, France), all very premature (<28 weeks of gestational age), were infected with E. cloacae. The strains were isolated and twelve of them, which exhibited several different characteristics, in particular ERIC-PCR profiles, were selected as shown in Table 1.

TABLE 1

| | | Susceptibility to 3^{rd} | |
|---|---|---|---|
| | | generation | |
| Isolate | Source of isolation | cephalosporins | ERIC-PCR profile |
| H1 | Blood | R | A |
| H2 | Blood | R | A |
| H7o | Blood | R | E |
| H7i | Blood | S | E |
| H8 | Blood | S | C |
| H9 | Blood | S | B |
| H10 | Blood | S | F |
| H11 | Blood | R | A |
| C12 | Cavum | S | H |
| C16 | Cavum | S | G |
| C17 | Cavum | S | E |
| C18 | Cavum | S | F |

To this end, the bacterial isolates were inoculated on a Columbia agar containing 5% of sheep blood ((bioMérieux, Marcy l'Étoile, France) and were incubated overnight at 37° C. Bacterial identification was confirmed by mass spectrometry (MALDI-TOF) (Brucker, Leipzig, Germany).

Antimicrobial susceptibility testing was performed using the agar disk diffusion method on Mueller-Hinton (MH, Bio-Rad, Hercules, Calif.) according to CLS138.

Enterobacterial repetitive intergenic consensus PCR (ERIC-PCR) was carried out in accordance with Duan et al. (Environ. Res. 109, 511-517; 2009). The ERIC band profiles obtained from agarose gel electrophoresis were used to define different profiles.

Composition of Molecular Species Present in Lipid A Regions.

The lipopolysaccharides were extracted from the cultured strains and the structures of their lipid A regions were analyzed using MALDI-TOF.

The bacteria were grown overnight at 37° C. in LB broth (Sigma) and the LPSs were isolated using the phenol/water method of Westphal and Jann (40). Briefly, the wet pellet of bacteria was shaken in a 45% aqueous phenol solution at 65° C. for 30 min, insoluble matter was removed from the cooled aqueous phase by centrifugation, and the clear extract was dialyzed in tap water until the phenol was eliminated, then dialyzed against distilled water. The extracts were subjected to enzymatic treatments (DNase, RNase and proteinase K) to remove DNA, RNA and proteins, then purified with acidified chloroform-methanol-water to remove contaminating phospholipids and lipoproteins. The LPSs were then washed in suspension in cold methanol, centrifuged (7000×g) and dried under a stream of air. Lipid A was prepared using the triethylamine-citrate method. Briefly, the LPS sample was suspended at a concentration of 10 μg/μl in a 0.01 M triethylamine-citrate solution (molar ratio 1:1, pH 3.6) and heated for 1 h at 100° C. The sample was then lyophilized and suspended in methanol. After centrifugation (7000×g for 10 min at 4° C.), lipid A was extracted with a mixture of chloroform:methanol:water (3:1.5:0.25; by volume) at a concentration of 10 μg/μl.

The molecular species present in this preparation were analyzed using an AXIMA performance matrix assisted laser desorption MALDI-TOF (matrix assisted) mass spectrometer (Shimadzu Biotech) (12BC, Université Paris Saclay, Gif sur Yvette, France). A suspension of lipid A (1 μg/μl) in chloroform:methanol:water (3:1.5:0.25, v:v:v) was desalted with a few grains of Dowex 50W-X8 (H+), 1 μl was deposited onto the target and mixed with 1 μl of a gentisic acid matrix (2,5-dihydroxybenzoic acid) (DHB from Fluka) suspended at 10 μg/μl in the same solvent or in 0.1 M of aqueous citric acid, and dried. Analyte ions were desorbed from the matrix with pulses of a 337 nm nitrogen laser. The spectra were obtained in negative ion mode at 20 kV, with the linear detector. Mass calibration was carried out with the AB SCIEX peptide mass standard kit or with a purified LPS sample and characterized by the structure of Bordetella pertussis and E. coli J5.

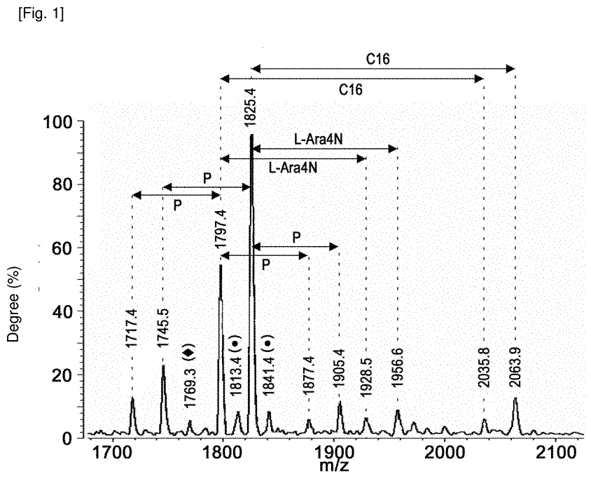

Like lipid A from other Gram-negative bacteria, the lipid A fragments isolated from the twelve selected strains of E. cloacae contained multiple molecular variants represented by multiple peaks in their mass spectra. Among the twelve lipids A, one of the most heterogeneous was that isolated from strain H7i (profile E with inducible cephalosporinase), with more than 13 significant peaks (13 molecular species) in its lipid A spectrum (FIG. 1). The spectrum contains a series of peaks (1360-1388, 1570-1598, 1797-1825, 1928-1956, 2035-2063) with a 28 mu distance between peaks, suggesting the presence of fatty acids of different lengths of two carbon atoms in the molecular variants of this lipid A. The composition of the molecular species corresponding to the various peaks is shown in Table 2.

TABLE 2

| | Peaks (calculated m/z) | | | | | | | | |
|---|---|---|---|---|---|---|---|---|---|
| Constituents | 1388.7 | 1599.1 | 1717.4 | 1745.5 | 1769.3 | 1783.4 | 1797.4 | 1811.4 | 1813.4 |
| Total fatty acids | 4 | 5 | | | | | | | 6 |
| C12 | | | 1 | | 2 | 1 | 1 | | 1 |
| C13 | | | | | | 1 | | 1 | |
| C14 | 1 | 2 | 1 | 2 | | | 1 | 1 | |
| C16 | | | | | | | | | |
| 3OH—C14 | 3 | 3 | 4 | 4 | 4 | 4 | 4 | 4 | 4 |
| 2OH—C14 | | | | | | | | | 1 |
| Phosphate | 2 | 2 | 1 | 1 | 2 | 2 | 2 | 2 | 2 |
| L-Ara4N | | | | | | | | | |
| | Peaks (calculated m/z) | | | | | | | | |
| Constituents | 1825.4 | 1841.4 | 1877.4 | 1905.4 | 1928.5 | 1956.6 | 2035.8 | 2063.9 | 2143.8 |
| Total fatty acids | | | | | | | | 7 | |
| C12 | | | 1 | | 1 | | 1 | | |
| C13 | | | | | | | | | |
| C14 | 2 | 1 | 1 | 2 | 1 | 2 | 1 | 2 | 2 |
| C16 | | | | | | | 1 | 1 | 1 |
| 3OH—C14 | 4 | 4 | 4 | 4 | 4 | 4 | 4 | 4 | 4 |
| 2OH—C14 | | 1 | | | | | | | |
| Phosphate | 2 | 2 | 3 | 3 | 2 | 2 | 2 | 2 | 3 |
| L-Ara4N | | | | | 1 | 1 | | | |

The base peak at m/z=1825 is due to a bisphosphorylated glucosamine disaccharide backbone substituted with two myristic fatty acids and four hydroxymyristic fatty acids (identified as 3OH—C14 by GC-MS). This means that in the homologous peak of the corresponding doublet, at m/z=1797, one of the two myristic acids (C14:0) is replaced by lauric acid (C12:0). The following doublets (1928-1956 and 2035-2063) are explained by the addition of palmitate (C16:0) and 4-amino-4-deoxy-L-arabinose (L-Ara4N), respectively, at m/z 1797 and 1825. In addition to the two dominant peaks of the spectrum (at m/z 1797.4 and 1825.4), small adjacent peaks at +16 Da (m/z 1813.4 and 1841.4, labeled • in FIG. 1) indicate the presence of another less abundant hydroxymyristate residue. GC/MS analysis (FIG. 4) indicated the presence in this LPS of traces of α-hydroxymyristic acid (2OH—C14), thus suggesting that a species of lipid A contains this fatty acid and explaining the small size of the observed peaks. This can be produced by an ortholog of the dioxygenase LpxO identified in S. enterica serovar typhimurium. This enzyme generates 2-hydroxymyristate by hydroxylation of the myristate transferred to lipid A by the acyltransferase MsB/LpxM17. Another small peak (m/z 1769.3) in this spectrum (shown by ♦ in FIG. 1) can be explained by the presence of a minor species containing two C12:0 secondaries instead of C12+C14 (m/z 1797.4) or C14.+C14 (m/z 1825.4) present in the major species.

Position of the Lipid A Aminoarabinose and Phosphate Residues.

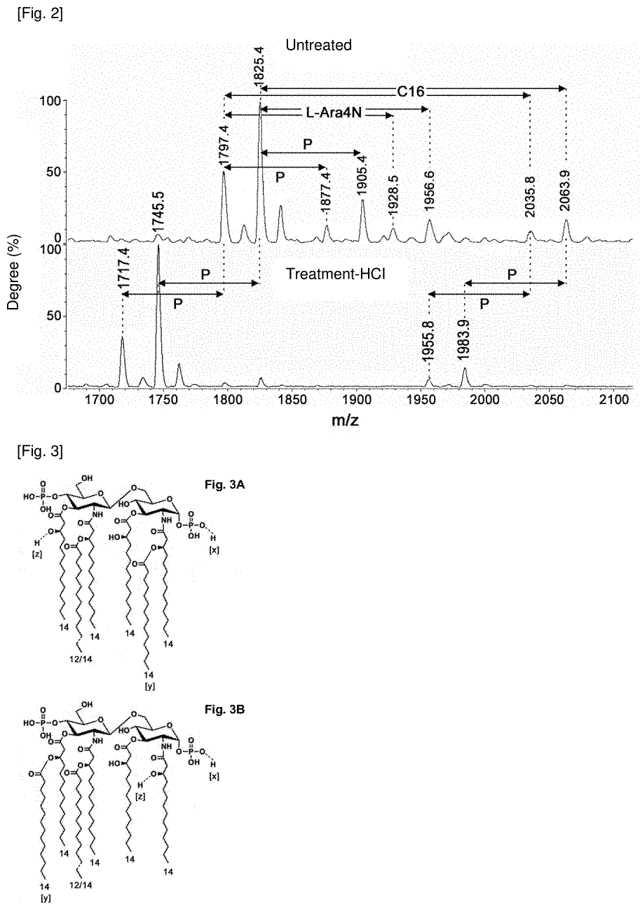

Monophosphorylated species of lipid A can be produced by acid hydrolysis (0.1 M HCl for 10 min at 100° C.). Labile bonds such as pyrophosphates and the acetal bond of the proximal glucosamine (phosphate bonded to C1) are hydrolyzed under these conditions. After such treatment of the lipid A of E. cloacae, peaks corresponding to bisphosphorylated and hexa-acylated species containing aminoarabinose (m/z 1928.5 and 1956.5) were completely absent from the MALTI-TOF spectrum (FIG. 2), such that the L-Ara4N group was not located on P4′ in the untreated bisphosphorylated species, because the L-Ara4N phosphate and phosphate bonds→4′-GlcN are both resistant. The loss of phosphoryl-aminoarabinose by mild acid hydrolysis proves that the L-Ara4N substituent is on the P1 phosphate of the bisphosphorylated species.

With respect to the phosphate groups, the presence of triphosphorylated molecules in untreated lipid A (m/z 1877.4 and 1905.4) indicates that a pyrophosphate must be present in these two molecular species. However, it should be noted that the molecules containing pyrophosphate (m/z 1877.4 and 1905.4) do not contain L-Ara4N and the molecules containing L-Ara4N (m/z 1928.5 and 1956.5) do not contain pyrophosphate. This suggests that during biosynthesis of the lipid A of E. cloacae, a third phosphate group or an L-Ara4N group is added to the P1 phosphate. It should be noted that the mild hydrolysis procedure used in this case did not induce extensive fatty acid cleavage because the hepta-acylated and monophosphorylated species (m/z 1955.8 and 1983.9) were still present after this hydrolysis.

Analysis of the Lipid A Acylation Profiles.

The sequential release of fatty acids linked to esters by mild alkaline treatment, used in previous studies (Silipo, A., et al. J. Lipid Res. 43, 2188-2195, 2002) generally provides valuable information concerning the positions of the various fatty acids on the lipid A backbone.

According to these studies, the secondary fatty acids of the C2′ and C2 groups (in particular C2′) are the most resistant, those of the C3 group (primary or secondary) are the most labile, with the substitutions of the C3′ group exhibiting intermediate behaviors. In addition, the secondary acids are more resistant than the primary fatty acids to alkaline treatments. In order to analyze the fatty acid acylation profiles in the LPS of E. cloacae, lipid A H7i was treated with 28% NH₄OH at 50° C. for 30 min, 1 h or 3 h.

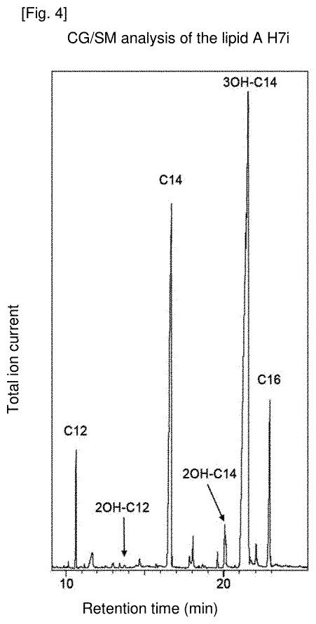

After this step, the complete structures of the main molecular species present in the lipid A complex of E. cloacae can now be proposed (FIG. 3). Four major and structurally different molecular species are present. In two of them (general structure A), a C14:0 secondary is at position 2, while in the other two (general structure B) it is at position 3′. Additionally, when present as a minor substituent, a palmitic group (C16:0) is at position 3′ in structure A and at position 2 in structure B.

Similarities and Differences Between the Lipid A Spectra of the Selected E. cloacae Strains. Comparison of the Lipids.

The spectra of the twelve strains of E. cloacae selected during this first step indicate that some peaks are not always present and that small additional peaks are sometimes detectable (Table 3).

TABLE 3

| | Source of isolation | | | | | | | | | | | |
|---|---|---|---|---|---|---|---|---|---|---|---|---|
| | Cavum | | | | Blood | | | | | | | |
| | Strain designation | | | | | | | | | | | |
| | C17 | C18 | C16 | C12 | H1 | H2 | H11 | H9 | H8 | H7o | H7i | H10 |
| | ERIC-PCR profile | | | | | | | | | | | |
| | E | F | G | H | A | A | A | B | C | E | E | F |
| | Expression AmpC* | | | | | | | | | | | |
| | i | i | i | i | o | o | o | i | i | o | I | i |
| m/z (calculated) | Peaks present in the spectrum | | | | | | | | | | | |
| 1388.7 | + | | | + | | | | | + | + | | + |
| 1599.1 | | | | | | | | | | | | + |
| 1717.4 | + | | + | + | | | | | | | + | |
| 1745.5 | + | + | + | + | | + | | | + | + | + | + |
| 1769.3 | + | + | | + | | | | | + | + | + | |
| 1783.4 | | | | + | | | | | | | | |
| 1797.4 | + | + | + | + | + | + | + | + | + | + | + | + |
| 1811.4 | | | | + | | + | | | + | | | |
| 1813.4 | | | | | | | | | | + | + | + |
| 1825.4 | + | + | + | + | + | + | + | + | + | + | + | + |
| 1841.4 | | | | | + | | | | | + | + | + |
| 1877.4 | | + | | + | | | | + | + | + | + | + |
| 1905.4 | | + | + | + | + | + | | + | + | + | + | + |
| 1928.5 | | + | | | + | + | + | + | + | + | + | + |
| 1956.6 | | + | | | + | + | + | + | | + | + | + |
| 2035.8 | + | + | | + | | + | | + | + | + | + | + |
| 2063.9 | + | + | + | + | + | + | + | + | + | + | + | + |
| 2143.8 | | + | | | | | | | + | | | + |

A tetraacylated (three hydroxymyristic acids and one myristic acid) and bisphosphorylated (peak at m/z=1388.7) glucosamine disaccharide is present in five strains (C17, C12, H8, H7o and H10), but it is absent from the other seven.

The tetraacylated molecular species observed in these five strains of E. cloacae contains three 3OH—C14 and one C14:0. The formation of this tetra-acylated species is most likely due to the loss, by enzymatic cleavage, of a myristoxy-myristoyl residue from the hexa-acylated form of lipid A at m/z 1825.4. Such cleavage requires the general structure B displayed in FIG. 3, which carries a C14:0 secondary on 3′. No correlation was found between this enzymatic cleavage detected in only five strains (C17, C12, H8, H7o and H10) and the ERIC-PCR profiles of these strains (profiles E, H, C, E and F, respectively).

The presence of other small peaks represents a second type of structural variation between the twelve strains. The small peaks at m/z 1813.4 and 1841.4 mentioned above in the spectrum of the H7i strain (presence of an α-hydroxymyristic acid) were actually present in four strains: H1, H7o, H7i and H10 (Table 3). They are due to a 2-hydroxymyristic acid (2OH—C14) that replaces the C14:0 secondary at positions 2 (FIG. 3A) or 3′ (FIG. 3B). The same C14:0 can also be replaced by C13:0 (peak at 1811.4 in strains C12, H2 and H8) or by C12:0 (peak at m/z 1769.3 in strains C17, C18, C12, H8, H7o and H7i) (Table 3 and FIG. 3).

Fatty Acid Composition of the LPSs Analyzed by Gas Chromatography/Mass Spectrometry.

The LPS samples (200 μg) were incubated at 85° C. for 15 to 18 h (but this time can be significantly reduced) in 600 μl of an anhydrous methanol/acetyl chloride mixture (10/1.5 in volumes) containing 4 μg of arachidic (eicosanoic) acid (C20) used as an internal standard. After this transmethylation reaction, the methanol was evaporated in a vacuum at room temperature, the resulting fatty acid methyl esters were extracted in ethyl acetate (600 μl) and the solvent was evaporated at room temperature under a stream of nitrogen. The material was dissolved in 50 μl of ethyl acetate and the solution (1-5 μl) was analyzed by gas chromatography coupled with mass spectrometry (GC-MS) in a Shimadzu appliance (GCMS-QP2010SE). A Phenomenex capillary column (Zebron ZB-5MS, 30 m<0.25 mm<0.25 μm) was used with a temperature gradient of 50° C. to 120° C. (20° C./min) followed by a gradient from 120° C. to 250° C. (3° C./min) and finally a constant temperature (250° C. for 2 min). The fatty acids were identified by their mass spectra (NIST base) and their retention times compared to the fatty acid standards (Sigma-Aldrich).

Analysis of the fatty acid composition of the LPSs by gas chromatography/mass spectrometry (GC/MS) confirmed the results obtained by MALDI, in particular the presence of small amounts of tridecanoic and 2-hydroxymyristic groups in some LPSs (FIG. 4).

Traces of a 2-hydroxylauric group (2OH—C12), undetectable by MALDI, were also detected by GC/MS in the LPSs of strains C12, H1, H9, H7o, H7i and H10.

Further variations among the twelve isolates are produced by the absence of significant MALDI peaks characteristic of the most complete molecular species. This is the case for the absence of pyrophosphate (trisphosphoryl species at m/z 1877.4 and 1905.4) in strains C17 and H11, as well as the absence of L-Ara4N (peaks at m/z 1928.5 and 1956.6) in strains C17, C16 and C12 (Table 2).

No correlation was observed between the lipid A structures of the various strains, characterized by their mass spectra, and their other available characteristics, such as their ERIC-PCR profile (A to H), the source from which they were isolated (cavum, rectum or blood), or the expression of cephalosporinase (inducible or overproduced). For example, strains H7o and H7i (isolated from the same infant) have the same ERIC-PCR profile and very similar lipid A mass spectra, but differ in terms of the cephalosporinase expression. Conversely, strains H11 and H7o both have an overproduction of cephalosporinase, but exhibit different lipid A spectra and ERIC-PCR profiles (profiles A and E, respectively).

### Example 2: Correlation Between the Presence of 2-Hydroxymyristate and the Pathogenicity of the Strains of the E. cloacae Complex

Although all the selected strains carry the same major molecular species of lipid A, they differ in minor variations, such as the presence of an L-Ara4N or a pyrophosphate at position 1 and the presence of a 2-hydroxymyristate (2OH—C14) or a C13:0 replacing a myristate (FIG. 3). It is therefore possible to classify the strains as a function of the presence of molecular species in their lipid A that may or may not carry some of these four constituents. Using this method, the twelve strains can be grouped into six groups as shown in Table 4.

TABLE 4

| | L-Ara4N+/PP+ | L-Ara4N+/P− | L-Ara4N−/PP+ |
|---|---|---|---|
| 2OH-C14+/C13− | H1 (†); H7h (†) | | |
| | H7i (†); H10 (†) | | |
| 2OH-C14-/C13+ | H2; H8 (†) | | C12 |
| 2OH-C14−/C13− | H9; C18 | H11 | C17; C16 |

†: deceased infant;
L-Ara4N: 4-amino-4-deoxy-L-arabinose;
PP: pyrophosphate;
2OH-C14: 2-hydroxymyristic acid;
C13: tridecanoic acid.
″+″ indicates the presence and ″−″ the absence of the corresponding substituent.

Notably, the lethal strains are not randomly distributed in the 6 identified groups but belong to only two groups. This result may suggest that the presence of 2-hydroxymyristate (2OH—C14) (100% (3/3) death) and, to a lesser extent, of tridecanoate (C13) (1/3 death; 33%) clearly improves the pathological power of the strain compared to the strains without these fatty acids (0% death).

A larger number of bacteraemic and non-bacteraemic babies was then studied to verify the correlation between the lethality induced by these strains in these two population groups and a structural element of their LPS system. These two population groups of infants were selected to be homogeneous in terms of their other characteristics and, in particular, do not differ in terms of other risk factors that contribute to neonatal mortality but are not related to their bacterial carriage. To this end, the CRIB II index (for “Clinical Risk Index for Babies”) was used. The CRIB II index is a hospital grading system that is widely used in neonatal intensive care and that reflects the severity of the condition of hospitalized babies. The score takes into account several characteristics of newborns, including the birth weight, the sex, the gestational age, the temperature, the Apgar score at 5 min, the base excess, the maternal age and parity.

In order to obtain a homogeneous population of infants, 18 premature infants infected with the E. cloacae complex were first selected. Then, for each of these babies, a colonized infant with a similar CRIB II score was selected. Strains from 18 infected infants were isolated from the blood culture, while in 18 colonized infants, 9 strains were isolated from the cavum and 9 from the rectum (Table 5). Of the 36 infants, 13 died within one month of birth, including 12/18 in the group of infected infants and 1/18 in the group of colonized infants. Death occurred on average 10±8 days after birth.

The presence of 2-hydroxymyristate (2OH—C14) in the LPS, as determined by GC-MS, was associated with the detection of the IpxO gene in 8 strains (Table 5). In LPS H9, 2-hydroxymyristate (2OH—C14) was undetectable in the MALDI spectrum (absence of peaks at m/z 1813.4 and 1841.4) and almost absent by GC-MS analysis (0.5%) compared with the average amount of this constituent in other strains (4.3±1.4%). It was therefore considered that LPS H9 is devoid of 2OH—C14. Data relating to the number of deaths as a function of the presence or absence of 2OH—C14 in the corresponding LPSs was extracted from Table 5.

TABLE 5

| | Strain | | | | | |
|---|---|---|---|---|---|---|
| | | | Expression | | IpxO | Death of |
| Status of | Source of | | of AmpC | 2OH—C14 | gene | the baby |
| the baby | isolation | Designation | * | (%) ** | *** | **** |
| colonized | Cavum | C1 | i | 6.3 | + | + |
| | | C2 | i | 0 | − | − |
| | | C3 | i | 0 | − | − |
| | | C4 | i | 0 | − | − |
| | | C6 | i | 0 | − | − |
| | | C7 | i | 0 | − | − |
| | | C9 | i | 0 | − | − |
| | | C21 | i | 0 | − | − |
| | | C25 | i | 0 | − | − |
| | Rectum | C5 | o | 0 | − | − |
| | | C8 | o | 0 | − | − |
| | | C10 | o | 0 | − | − |
| | | C11 | o | 0 | − | − |
| | | C23 | o | 4.7 | + | − |
| | | C24 | o | 4.9 | + | − |
| | | C26 | o | 0 | − | − |
| | | C27 | i | 0 | − | − |
| | | C28 | i | 0 | − | − |
| infected | blood | H1 | o | 6.3 | − | + |
| | | H2 | o | 0 | − | − |
| | | H3 | o | 2.6 | − | + |
| | | H4 | o | 5.4 | + | + |
| | | H5 | i | 4.2 | + | + |
| | | H6 | o | 0 | − | + |
| | | H7 | o | 4.9 | + | + |
| | | H8 | i | 0 | − | + |
| | | H9 | i | 0.5 | − | − |
| | | H10 | i | 4.5 | + | + |
| | | H11 | o | 0 | − | − |
| | | H21 | i | 0 | − | − |
| | | H23 | i | 0 | − | + |
| | | H24 | o | 0 | − | + |
| | | H25 | i | 0 | − | − |
| | | H26 | o | 2.2 | + | − |
| | | H27 | i | 0 | − | + |
| | | H28 | i | 0 | − | + |

* inducible (i) or overproduced (o) cephalosporinase.
** expressed by the ratio: 2OH—C14 × 100/3OH—C14.
*** + detected; − not detected.
**** + baby died within the first month after birth; − baby survived.

Patients with E. cloacae sepsis carrying this 2OH—C14 marker in the LPS were compared with those lacking the marker. Patients with LPS carrying 2OH—C14 have a higher VIS score (vasoactive and inotropic drugs needed to maintain normal systemic blood pressure) (p=0.03) and are more likely to develop oliguria (p=0.02) than those without this LPS marker. A Fisher's exact test carried out on the data shows a significant dependence (p=0.0178) between the presence of 2OH—C14 in the LPSs of the E. cloacae complex and the mortality induced by these strains.

The deaths of 9 newborns were directly attributable to E. cloacae septic shock. Other causes of death were spontaneous intestinal perforation (n=2) and Haemophilus influenzae pneumonia (n=1), Bacteroides fragilis infection (n=1), necrotizing enterocolitis (n=1), and severe bronchopulmonary dysplasia (n=1). Consequently, the data corresponding to deaths not attributable to E. cloacae septic shock must be removed from the statistical analysis. A survival analysis according to Kaplan-Meier shows a highly significant imputability on mortality of the presence of LPS carrying 2OH—C14 (p=0.007 using the logrank test).

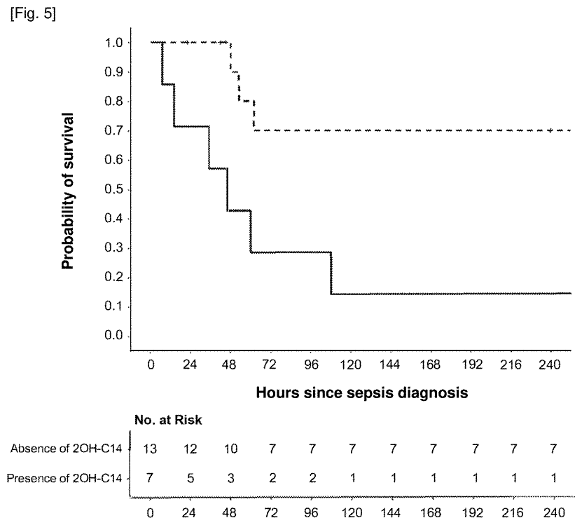

The results are shown in FIG. 5. On the diagram, the solid line curve corresponds to the strains for which 2-hydroxymyristic acid is present in the LPS. The dashed curve corresponds to the strains for which 2-hydroxymyristic acid is absent in the LPS.

The risk ratio of death in patients infected with Enterobacter cloacae whose LPS has 2OH—C14 is 5.55.

### Example 3: E. cloacae Complex Polymyxin B Survival Assay

Strains of the Enterobacter complex were selected.

The Enterobacter strains preserved in a tube frozen with 30% glycerol are transplanted in a planktonic culture in 20 ml of an LB culture medium. After incubation at 37° C. overnight, 1 ml of the culture diluted to 10⁷ is brought into contact with increasing concentrations of polymyxin B (1 μg/ml, 3 μg/ml and 10 μg/ml). A tube without antibiotic is added as a control.

All the tubes are incubated at 37° C. with shaking for an additional 4 hours. The cultures are then diluted and spread on LB agar Petri dishes.

The dishes are incubated at 37° C. overnight and the colonies are counted.

Percentage survival is computed as follows:

(CFU polymyxin B/CFU control)×100

5 strains of E. cloacae complex expressing 2-hydroxymyristate (2HM) and 5 strains of E. cloacae complex not expressing 2-hydroxymyristate (2HM) were exposed to different concentrations of polymyxin B for 4 hours.

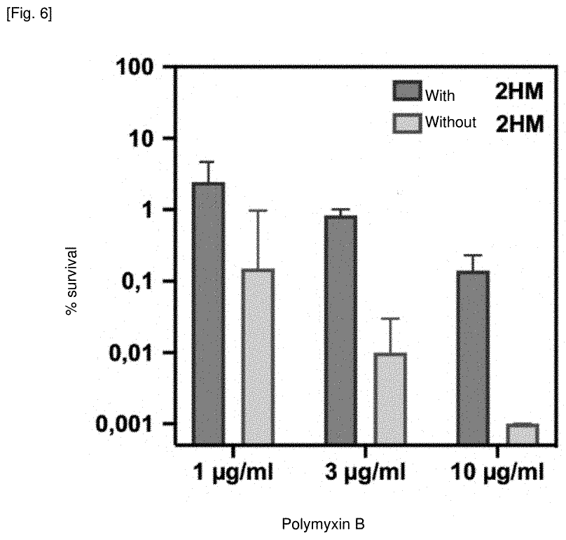

The results presented in FIG. 6 show that the presence of 2-OH myristate on Lipid A generates resistance to treatment with polymixin B.

### Example 4: Study of the Presence of lpxO

The insertion of 2-hydroxymyristate (2HM) into the Lipid A structure is under the control of an LpxO hydroxylase described in Salmonella. The lpxO gene exists within the bacterial chromosome sequences of several ECCs on GenBank.

Whole genome sequencing of the 20 strains of Enterobacter cloacae complex (ECC) was carried out using Illumina technology. An alignment of sequences via Clustal Omega between the lpxO sequences of our strains (n=5) and sequences available on GenBank from among the genomes of ECC and other Gram-negative bacteria was carried out. The search in the NGS sequences for a counterpart of lpxO found its presence in 5 of the 7 strains with 2HM. Within the genus Enterobacter, the lpxO sequences of the various species are more than 80% identical except for all the sequences available in E. hormaechei, which had only 60% identity with the other Enterobacter. While the lpxO sequences from the same Enterobacter species vary very little (for example, from 96% to 100% in E. bugandensis and from 99 to 100% in E. cloacae), all the lpxO sequences of E. hormaechei are 100% identical and appear to originate from a plasmid.

1.-12. (canceled)

13. A method for predicting the pathogenicity of a strain of Gram-negative bacteria of the Enterobacteriaceae family, wherein the amount of 2-hydroxymyristate and/or 2-hydroxymyristic acid present in the lipopolysaccharide of the bacteria is measured, said amount of 2-hydroxymyristate and/or 2-hydroxymyristic acid is compared with a reference value, and wherein it is concluded that the strain is pathogenic if the amount of 2-hydroxymyristate and/or 2-hydroxymyristic acid is greater than the reference value.

14. The method as claimed in claim 13, wherein the bacterium belongs to the genus Enterobacter, Escherichia, Klebsiella, Pseudomonas, Salmonella, Serratia or Yersinia.

15. The method as claimed in claim 13, wherein the bacterium belongs to the genus Enterobacter, Escherichia, Pseudomonas, Serratia or Yersinia.

16. The method as claimed in claim 13, wherein the Gram-negative bacteria are bacteria of the genus Enterobacter belonging to the species of the Enterobacter cloacae complex.

17. The method as claimed in claim 13, wherein the amount of 2-hydroxymyristate is compared with the amount of 3-hydroxymyristate present in the lipopolysaccharide of the bacteria, and in that if the ratio of 2-hydroxymyristate to 3-hydroxymyristate is greater than 0.01, it is concluded that the strain is pathogenic.

18. The method as claimed in claim 16, wherein the method is a method for predicting the resistance of strains of Enterobacter cloacae to antibiotics and/or to the responses of the host to the antimicrobial peptides produced by the host.

19. The method as claimed in claim 13, wherein the bacteria are present in a biological sample.

20. The method as claimed in claim 19, wherein the biological sample originates from a mammal, preferably from a human being, and is selected from blood, serum, plasma, cerebrospinal fluid (LCR), ascites, pleural fluid, mucus, stools, or a mucocutaneous sample.

21. The method as claimed in claim 13, further comprising a prior step of isolating the bacteria from a sample.

22. The method as claimed in claim 13, wherein the reference value is equal to 0.

23. A method of predicting the pathogenicity and/or of virulence of a Gram-negative bacterial strain using a 2-hydroxymyristic acid ester as a marker thereof, comprising measuring a concentration of 2-hydroxymyristic acid ester in a Gram-negative bacterial strain selected from bacteria of the genus Enterobacter, Escherichia, Klebsiella, Pseudomonas, Salmonella, Serratia and/or Yersinia, (in particular of Enterobacter cloacae complex).

24. A kit for diagnosing the pathogenicity of a strain of Enterobacter cloacae complex, comprising means for measuring the concentration of 2-hydroxymyristate in the LPS of a bacterial isolate.
